(19) United States
(12) Patent Application Publication
(10) Pub. No.: US 2013/0210089 A1
(43) Pub. Date: Aug. 15, 2013

(54) PROCESS FOR THE PRODUCTION OF SUGARS FROM LIGNOCELLULOSIC BIOMASS PRE-TREATED WITH A MIXTURE OF HYDRATED INORGANIC SALTS AND METALLIC SALTS

(71) Applicants: Christophe Vallee, Sassenage (FR); Didier Morvan, Mornant (FR); Emmanuel Pellier, Tupin Semons (FR); Helene Olivier-Bourbigou, St Genis Laval (FR)

(72) Inventors: Christophe Vallee, Sassenage (FR); Didier Morvan, Mornant (FR); Emmanuel Pellier, Tupin Semons (FR); Helene Olivier-Bourbigou, St Genis Laval (FR)

(73) Assignee: IFP ENERGIES NOUVELLES, Rueil Malmaison Cedex (FR)

(21) Appl. No.: 13/811,676

(22) PCT Filed: Jul. 19, 2011
(86) PCT No.: PCT/FR2011/000426

§ 371 Date: Apr. 4, 2013

(30) Foreign Application Priority Data
Jul. 23, 2010 (FR) 10/03092

(51) Int. Cl. C12P 19/02

(52) U.S. Cl. CPC C12P 19/02
USPC 435/105

(57) ABSTRACT

The present invention concerns a process for the conversion of lignocellulosic biomass into sugars, comprising at least three steps. The first step is a step for cooking the lignocellulosic biomass in the presence of at least one hydrated inorganic salt mixed with at least one other metallic salt. The second step is a step for separating at least one solid fraction which has undergone the cooking step, and the third step is a step for enzymatic hydrolysis of said solid fraction to convert the polysaccharides into monosaccharides. The sugars obtained thereby can then be fermented into alcohols.

### FIELD OF THE INVENTION

The present invention falls into the context of a “second generation” process for the production of alcohol from lignocellulosic biomass.

### PRIOR ART

Faced with an increase in pollution and climatic warming, many studies are currently being carried out to use and optimize renewable bioresources such as lignocellulosic biomass.

Lignocellulosic biomass is composed of three principal polymers: cellulose (35% to 50%), hemicellulose (23% to 32%), which is a polysaccharide essentially constituted by pentoses and hexoses, and lignin (15% to 25%), which is a polymer with a complex structure and a high molecular weight deriving from copolymerization of phenylpropenoic alcohols. These various molecules are responsible for the intrinsic properties of the plant wall and organize themselves into a complex tangle.

Cellulose, which is in the majority in this biomass, is thus the most abundant polymer on Earth and therefore has the greatest potential for forming materials and biofuels. However, the potential of cellulose and its derivatives has not so far been able to be exploited to its fullest extent, primarily because cellulose is difficult to extract. In fact, this step is rendered difficult by the very structure of the plants. Particular technological problems which have been identified which are linked to the transformation of cellulose are its accessibility, its crystallinity, its degree of polymerization, and the presence of hemicellulose and of lignin. Thus, it is vital to develop novel methods for pre-treating the lignocellulosic biomass in order to gain easier access to the cellulose and allow it to be transformed.

The production of biofuel is an application necessitating a pre-treatment of the biomass. In fact, second generation biofuel uses vegetable or agricultural waste such as wood, wheat straw, or planting with a high growth potential, such as miscanthus, as a feedstock. This starting material is perceived as an alternative, durable solution which has little or no impact on the environment and is cheap and widely available; it is thus a strong candidate for the production of biofuels.

The principle of the process for the conversion of lignocellulosic biomass into biofuel employs a step for enzymatic hydrolysis of the cellulose contained in the plant material to produce glucose. This glucose is then fermented into the biofuel, ethanol.

However, the cellulose contained in the lignocellulosic biomass is particularly refractory to enzymatic hydrolysis, in particular because cellulose is not directly accessible to enzymes. In order to deal with this refractory nature, a step for pre-treatment upstream of the enzymatic hydrolysis is necessary. A number of methods for treating cellulose-rich materials exist for improving the subsequent enzymatic hydrolysis step; they are chemical, enzymatic or microbiological in nature.

Examples of such methods are: steam explosion, the organosolv process, hydrolysis with dilute or concentrated acid, or the AFEX (ammonia fibre explosion) process. Such techniques can still be improved upon; in particular, they are as yet too expensive, there are problems with corrosion, the yields are low and difficulties are encountered in scaling up to industrial levels (F. Talebnia, D. Karakashev, I. Angelidaki Biores. Technol. 2010, 101, 4744-4753).

For a number of years, a novel type of pre-treatment consisting of using ionic liquids has been studied. Ionic liquids are salts constituted uniquely of liquid ions at temperatures of 100° C. or less which can be used to obtain highly polar media. Hence, they are used as solvents or as reaction media for the treatment of cellulose or lignocellulosic materials (WO 05/17252; WO 05/23873). However, in similar manner to the other pre-treatments, with ionic liquids, problems arise with high costs linked to the price of the ionic liquids and to the fact that they are often difficult to recycle.

It appears to be necessary to limit the costs of pre-treatment, in particular by using readily available, cheap reagents. This constitutes the context of the present invention.

### SUMMARY OF THE INVENTION

The present invention concerns a process for the conversion of lignocellulosic biomass into sugars, comprising at least three steps. The first step is a step for cooking the lignocellulosic biomass in a medium comprising at least one hydrated inorganic salt mixed with at least one other metallic salt. The second step is a step for separating at least one solid fraction which has undergone the cooking step, and the third step is a step for enzymatic hydrolysis of said solid fraction in order to convert the polysaccharides into monosaccharides. The sugars obtained thereby can then be fermented into alcohols.

### DESCRIPTION OF THE FIGURES

FIG. 1 represents the kinetics of the enzymatic hydrolysis of wheat straw in the process of the present invention, using a step for cooking the biomass in the presence of LiCl.H₂O(98%)/ZnCl₂.2.5H₂O(2%) at 140° C.

FIG. 2 represents the kinetics of the enzymatic hydrolysis of wheat straw in the process of the present invention, using a step for cooking the biomass in the presence of LiCl.2H₂O(10%)/ZnCl₂.2.5H₂O(90%) at 80° C.

FIG. 3 represents the kinetics of the enzymatic hydrolysis of wheat straw in the process of the present invention, using a step for cooking the biomass in the presence of NaCl.6H₂O(10%)/ZnCl₂.2.5H₂O(90%) at 80° C.

FIG. 4 represents the kinetics of the enzymatic hydrolysis of wheat straw in the process of the present invention, using a step for cooking the biomass in the presence of NaCl.6H₂O(10%)/ZnCl₂.2.5H₂O(90%) recycled at 80° C.

FIG. 5 represents the kinetics of the enzymatic hydrolysis of wheat straw in the process of the present invention, using a step for cooking the biomass in the presence of LiCl.2H₂O(10%)/FeCl₃.6H₂O(90%) at 60° C.

FIG. 6 represents the kinetics of the enzymatic hydrolysis of wheat straw in the process of the present invention, using a step for cooking the biomass in the presence of LiCl.2H₂O(20%)/FeCl₃.6H₂O(80%) at 60° C.

FIG. 7 represents the kinetics of the enzymatic hydrolysis of a native wheat straw which has not undergone any pre-treatment.

FIG. 8 represents the kinetics of the enzymatic hydrolysis of wheat straw when it is pre-treated by steam explosion prior to enzymatic hydrolysis.

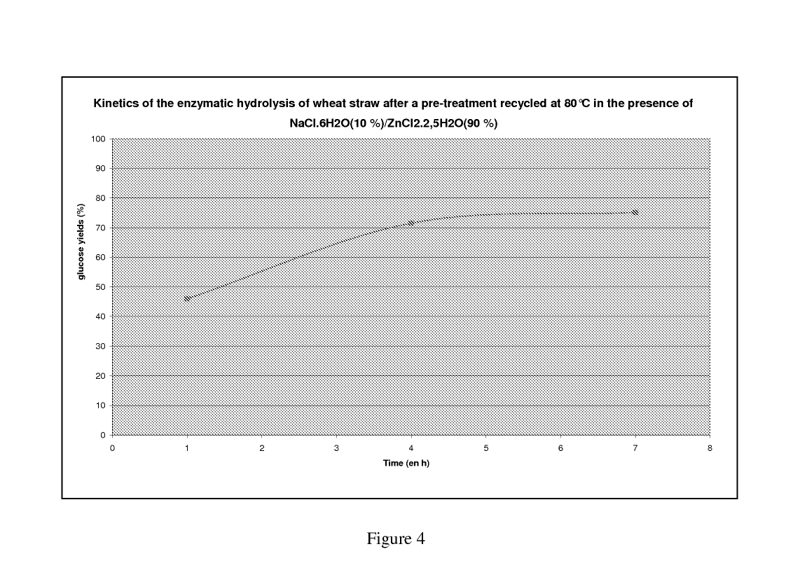

### DETAILED DESCRIPTION OF THE INVENTION

The process for the conversion of lignocellulosic biomass into monosaccharides of the present invention comprises at least:
a) a step for cooking the biomass in the presence or in the absence of an organic solvent in a medium comprising at least one hydrated organic salt with formula (1):

MXₙ.n′H₂O
in which X is an anion and M is a metal selected from groups 1 and 2 of the periodic classification of the elements, n is a whole number equal to 1 or 2 and n′ is in the range 0.5 to 6;
mixed with at least one other metallic salt, which may or may not be hydrated, with general formula (2):

M′Yₘ.mH₂O
in which Y is an anion, which may be identical to or different from X, and m′ is a metal selected from groups 3 to 13 of the periodic classification of the elements, m is a whole number in the range 1 to 6 and m′ is in the range 0 to 6;
b) a step for separating a solid fraction which has undergone the cooking step;
c) a step for enzymatic hydrolysis of said solid fraction.

This process can be used to transform lignocellulosic biomass into fermentable sugars in excellent yields. In addition, it has the advantage of using cheap reagents, which are widely available and which can be recycled, meaning that the cost of pre-treatment is low, in particular compared with a process using ionic liquids. This technology is also simple to implement and it is envisaged that scaling up to an industrial level should be easy.

Thanks to the process of the present invention, the transformation of lignocellulosic biomass into fermentable sugars is carried out with an excellent yield. The cooking step carried out in the process of the present invention can be used to reduce the duration of enzymatic hydrolysis compared with the processes described in the prior art. The process of the present invention can be used to obtain high glucose yields with short cooking periods, resulting in a large gain in terms of productivity of the equipment, since it becomes possible to use smaller quantities of enzymes and/or to reduce the size of the enzymatic hydrolysis tanks.

The process of the invention can be used to carry out the step for cooking of the lignocellulosic biomass at moderate temperatures in the absence of pressure; this constitutes a major gain in terms of energy costs.

Preferably, the medium in which the cooking step is carried out is constituted by one or more hydrated inorganic salts with formula (1) mixed with at least one other metallic salt, which may or may not be hydrated, having formula (2).

This cooking step is carried out in the presence or absence of an organic solvent.

The lignocellulosic biomass or lignocellulosic materials employed in the process of the invention is obtained from wood (deciduous or coniferous), green or treated, agricultural by-products such as straw, plant fibres, forestry crops, residues from alcohol-producing plants, sugar-producing plants and cereal-producing plants, residues from the paper industry, or products from the transformation of cellulosic or lignocellulosic materials. The lignocellulosic materials may also be biopolymers and are preferably rich in cellulose.

Preferably, the lignocellulosic biomass used is wood, wheat straw, wood pulp, rice straw or corn stalks.

In the process of the present invention, the various types of lignocellulosic biomass may be used alone or as a mixture.

In the cooking step, the lignocellulosic biomass is present in a quantity in the range 0.5% to 40% by weight of the total mass of the lignocellulosic biomass/hydrated inorganic salt/metallic salt mass, preferably in a quantity in the range 3% to 25% by weight.

Preferably, the anion X is a halide anion selected from Cl, F, Br and I, a perchlorate anion (ClO₄), a thiocyanate anion (SCN), a nitrate anion (NO₃) or an acetate anion (CH₃COO).

The metal M is preferably selected from lithium, magnesium, calcium, potassium and sodium.

Preferably, in the formula MXₙ.n′H₂O (1) for the hydrated inorganic salt, n′ is in the range 1 to 6.

In accordance with the present invention, the hydrated inorganic salt with formula (1) may be prepared in situ by associating a salt composed of a cation from groups 1 and 2 of the periodic classification of the elements and a carbonate, bicarbonate or hydroxide anion with an acid. The acid may be used pure or in aqueous solution.

Preferably, in the metallic salt with formula (2), the anion Y is selected from halides, nitrates, carboxylates, halogenocarboxylates, acetylacetonates, alcoholates, phenolates, optionally substituted, sulphates, alkylsulphates, phosphates, alkylphosphates, fluorosulphonates, alkylsulphonates, for example methylsulphonate, perfluoroalkylsulphonates, for example trifluoromethylsulphonate, bis(perfluoroalkylsulphonyl)amides, for example bis-trifluoromethylsulphonyl amide with formula N(CF₃SO₂)₂⁻, arenesulphonates, optionally substituted with halogen or alkylhalogen groups.

More preferably, the anion Y is selected from fluoride, chloride, bromide, acetate and triflate anions.

Preferably, the metal M′ is selected from iron, cobalt, nickel, copper, zinc, aluminium, indium and lanthanum.

Preferably, the metallic salt with general formula (2) is selected from FeBr₃, FeCl₃, FeCl₃.6H₂O, FeF₃, Fe(NO₃)₃, CoCl₂, CoCl₂.6H₂O, Ni(CH₃COO)₂.4H₂O, NiBr₂, NiCl₂, NiCl₂.6H₂O, Zn(CH₃COO)₂.2H₂O, ZnBr₂, ZnCl₂, ZnCl₂.2.5H₂O, AlBr₃, AlCl₃, AlCl₃.6H₂O, LaCl₃, LaCl₃.6H₂O.

The mole fraction of the hydrated inorganic salt with formula (1) in the mixture of at least one hydrated inorganic salt with formula (1) and at least one metallic salt with formula (2) may be in the range 0.05 to 1.

The step for cooking the biomass may be carried out in a medium constituted by a mixture of different hydrated inorganic salts with formula (1) and/or different metallic salts with formula (2).

Examples of molar compositions for mixtures of hydrated inorganic salts and metallic salts which may be used for the cooking step in accordance with the present invention that may be cited are as follows:

LiCl.H₂O(98%)/ZnCl₂.2.5H₂O(2%),

LiCl.2H₂O(10%)/ZnCl₂.2.5H₂O(90%),

NaCl.6H₂O(10%)/ZnCl₂.2.5H₂O(90%),

LiCH₃COO.3H₂O(10%)/ZnCl₂.2.5H₂O(90%),

LiCl.2H₂O(20%)/ZnCl₂.2.5H₂O(80%),

NaCl.6H₂O(20%)/ZnCl₂.2.5H₂O(80%),

LiCl.2H₂O(10%)/FeCl₃.6H₂O(90%),

NaCl.6H₂O(10%)/FeCl₃.6H₂O(90%),

LiCH₃COO.3H₂O(10%)/FeCl₃.6H₂O(90%),

LiCl.2H₂O(20%)/FeCl₃.6H₂O(80%),

NaCl.6H₂O(20%)/FeCl₃.6H₂O(80%).

In the process of the present invention, several successive cooking steps may be carried out in a medium constituted by at least one hydrated inorganic salt with formula (1) mixed with at least one metallic salt with formula (2).

Preferably, the cooking temperature is preferentially in the range −20° C. to 250° C., preferably in the range 20° C. to 160° C. Highly preferably, the cooking temperature does not exceed 100° C.

The cooking period is in the range 0.5 minutes to 168 h, preferably in the range 5 minutes to 4 h and more preferably in the range 20 minutes to 2 h.

The step for cooking the lignocellulosic biomass in accordance with the present invention may be carried out in the presence of an organic solvent. The organic solvent may be selected from alcohols such as methanol, ethanol, n-propanol, isopropanol, n-butanol, isobutanol or tert-butanol, diols and polyols such as ethanediol, propanediol or glycerol, amino alcohols such as ethanolamine, diethanolamine or triethanolamine, ketones such as acetone or methyl ethyl ketone, carboxylic acids such as formic acid or acetic acid, esters such as ethyl acetate or isopropyl acetate, dimethylformamide, dimethylacetamide, dimethylsulphoxide, acetonitrile, and aromatic solvents such as benzene, toluene, xylenes or alkanes.

In another implementation, the step for cooking the lignocellulosic biomass of the present invention may be carried out in the absence of an organic solvent.

The second step b) of the process of the invention consists of separating a solid fraction which has undergone the cooking step a) described above. This separation is generally carried out by adding at least one anti-solvent which causes precipitation of the solid fraction.

Separation of this precipitated solid fraction and a liquid fraction containing the hydrated inorganic salt, the metallic salt and the anti-solvent may be carried out using the usual solid-liquid separation techniques. As an example, the solid fraction which has undergone the cooking step may be separated by filtration or by centrifuging.

The solid fraction may optionally undergo supplemental treatments prior to the enzymatic hydrolysis step. These supplemental treatments may in particular be intended to eliminate traces of hydrated inorganic salts and/or metallic salts from the solid fraction which has undergone the cooking step. These supplemental treatments may, for example, be washes, carried out with the anti-solvent, with water or with any other stream of the process.

The liquid obtained after washing contains the fluid used for the supplemental treatment and traces of hydrated inorganic salts and/or metallic salts. This liquid may advantageously be recycled for use during the separation step, thereby providing for better recovery of the salts and higher purity of the solid fraction while at the same time limiting the consumption of anti-solvent or other washing fluid.

The solid fraction may optionally be dried or compressed in order to increase the percentage of dry matter contained in the solid.

The anti-solvent used is a solvent or a mixture of solvents selected from water, alcohols such as methanol, ethanol, n-propanol, isopropanol, n-butanol, isobutanol or tert-butanol, diols and polyols such as ethanediol, propanediol or glycerol, amino alcohols such as ethanolamine, diethanolamine or triethanolamine, ketones such as acetone or methyl ethyl ketone, carboxylic acids such as formic acid or acetic acid, esters such as ethyl acetate or isopropyl acetate, dimethylformamide, dimethylacetamide, dimethylsulphoxide, and acetonitrile.

Preferably, the anti-solvent is selected from water, methanol and ethanol.

Highly preferably, the anti-solvent is water.

The hydrated inorganic salt and the metallic salt contained in the liquid fraction may be separated from the anti-solvent and recycled, for example for use during the cooking step. This separation may be carried out using any of the processes known to the skilled person such as, for example, evaporation, precipitation, extraction, passage over an ion exchange resin, electrodialysis, chromatographic methods, solidification of the hydrated inorganic salt and of the metallic salt by reducing the temperature or adding a third substance, and reverse osmosis.

The liquid fraction containing the hydrated inorganic salt, mixed with the metallic salt, and the anti-solvent may also contain products derived from the biomass. As an example, the liquid fraction may contain hemicellulose (or products derived from hemicellulose) and lignin.

The products derived from the biomass contained in liquid fraction may be separated before or after separating the hydrated inorganic salt as a mixture with the metallic salt-anti-solvent. The products derived from the biomass may, for example, be extracted by adding a solvent which is not miscible with the hydrated inorganic salt mixed with the metallic salt or with the mixture of a hydrated inorganic salt mixed with the metallic salt-anti-solvent. The products derived from the biomass may also be precipitated by modifying the conditions (temperature, pH, etc.) or by adding a third substance.

The hydrated inorganic salt mixed with the metallic salt which is thus recovered may be purified by heating to high temperatures, over 400° C., to eliminate the organic products derived from the biomass that may still be present by combustion.

The third step c) of the process of the invention is the step for enzymatic hydrolysis of the solid fraction obtained in step b).

The solid fraction, which below is also termed the substrate, undergoes enzymatic hydrolysis in order to convert the polysaccharides into monosaccharides under conditions which are normally applicable in such conversion processes.

Typically, the pre-treated substrate is placed in an aqueous medium in order to obtain dry matter concentrations in the range 0.5% to 40%, preferably in the range 1% to 20% by weight.

The enzymatic hydrolysis is carried out under mild conditions, at a temperature of the order of 40° C. to 60° C., at a pH in the range 4.5 to 5.5. Highly preferably, the pH is in the range 4.8 to 5.2. If the pH has to be adjusted, it is carried out prior to the enzymatic hydrolysis step when the pre-treated substrate is placed in an aqueous medium, in particular by adding a buffer solution.

The enzymatic hydrolysis is carried out using enzymes produced by a microorganism. Microorganisms such as fungi belonging to the genuses Trichoderma, Aspergillus, Penicillum or Schizophyllum, or anaerobic bacteria belonging to the genus Clostridium, for example, produce such enzymes, in particular containing cellulases and hemicellulases, which are adapted to the intense hydrolysis of cellulose and hemicelluloses.

The duration of the hydrolysis step c) is in the range 1 h to 150 h, preferably in the range 2 h to 72 h, preferably in the range 4 h to 24 h.

At the end of the enzymatic hydrolysis step, the glucose formed is soluble in water while any unconverted cellulose, lignin or other products remain insoluble. The aqueous glucose solution is recovered by filtering.

The monosaccharide obtained thereby is readily transformed into alcohol by fermentation with yeasts such as Saccharomyces cerevisiae, for example. The fermentation must that is obtained is then distilled in order to separate the wash from the alcohol produced.

In accordance with one implementation of the invention, the enzymatic hydrolysis step is carried out simultaneously with fermentation.

### EXAMPLES

The substrate used in these examples was wheat straw. Compositional analysis, carried out in accordance with the NREL TP-510-42618 protocol, indicated that the composition of the unrefined substrate was as follows (as the percentage of dry matter): 37% cellulose, 28% hemicellulose and 20% lignin.

In the examples below, the compositions are given as mole fractions.

Examples 1 to 5 are in accordance with the invention. Example 6 is given by way of comparison, with the enzymatic hydrolysis being carried out on a native wheat straw. Example 7 is a comparative example, with the pre-treatment that was carried out consisting of steam explosion, which is in current use as one of the best techniques for obtaining fermentable sugars.

### Example 1

### Pre-Treatment of Wheat Straw with LiCl.H₂O(98%)/ZnCl₂.2.5H₂O(2%) at 140° C. and Enzymatic Hydrolysis

38 g of LiCl.H₂O(98%)/ZnCl₂.2.5H₂O(2%) and 2 g of wheat straw (500-1000 μm) were placed in a 170 mL flask and mechanically paddle stirred at 140° C. for 1 h in a Tornado© stirrer provided with a 6-position carousel (Radleys).

After this pre-treatment (step a) of the process of the invention), heating was halted and 80 mL of distilled water was rapidly added to the mixture: the pre-treated straw precipitated out. The suspension containing the mixture of salts, water and biomass was placed in a centrifuge tube and stirred at 9500 rpm for 10 minutes. The supernatant containing the mixture of salts was then separated from the solid. The operation was carried out three times by adding 80 mL of distilled water to the solid portion still present in the centrifuging tube.

7.4 g of solid with a dry matter content of 19% was recovered.

The solid recovered after precipitation and washing underwent enzymatic hydrolysis. Half of the recovered solution was placed in a 100 mL Schott flask. 5 mL of acetate buffer, and 10 mL of a 1% by weight solution of NaN₃ in water were added then made up to 100 g with distilled water. This solution was then left overnight at 50° C. for “activation”, at an agitation rate of 550 rpm in a STEM heater/shaker reaction station. Next, known quantities of enzyme were added to the solution:
XL508 Cellulases, 10 FPU per gram of dry matter;
NOVOZYM 188 β-glucosidases, 25 CBU per gram of dry matter.

The solution was then stirred at 400 rpm at 50° C., still in the heater/shaker reaction station, and samples were taken after 1 h, 4 h and 7 h. These samples were placed in centrifuge tubes and rapidly placed in an oil for 10 minutes at a temperature of 103° C. to neutralize the enzymatic activity. The centrifuge tubes were stored in a refrigerator at 4° C. to await glucose measurement. They were then diluted 5-fold with distilled water before being assayed using an Analox GL6 analytical apparatus known as a glucostat, which measures the concentration of glucose in aqueous solutions by enzymatic assay.

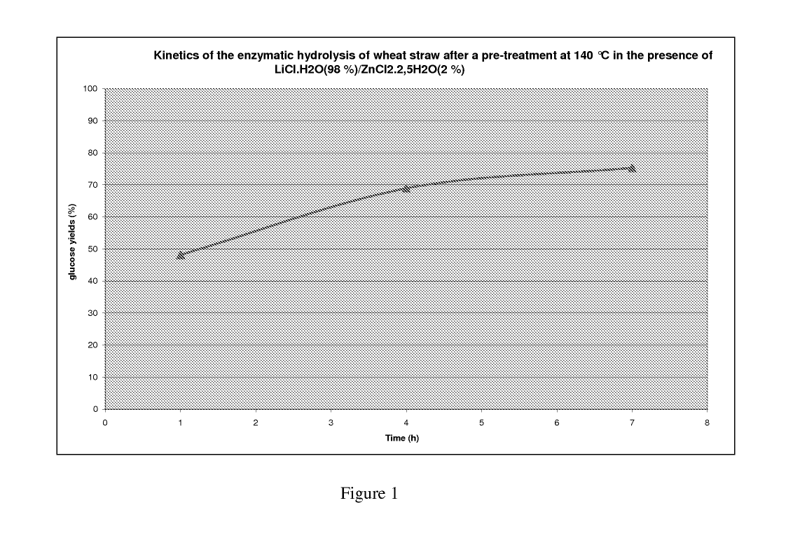

The results of the enzymatic hydrolysis are shown in FIG. 1. They are expressed as the glucose yield, defined as the ratio of the concentration of glucose in the solution to its maximum theoretical concentration as a function of the quantity of cellulose in the substrate. This glucose yield thus represents the percentage of cellulose effectively transformed into glucose.

### Example 2

### Pre-Treatment of Wheat Straw with LiCl.2H₂O(10%)/ZnCl₂.2.5H₂O(90%) at 80° C. and Enzymatic Hydrolysis

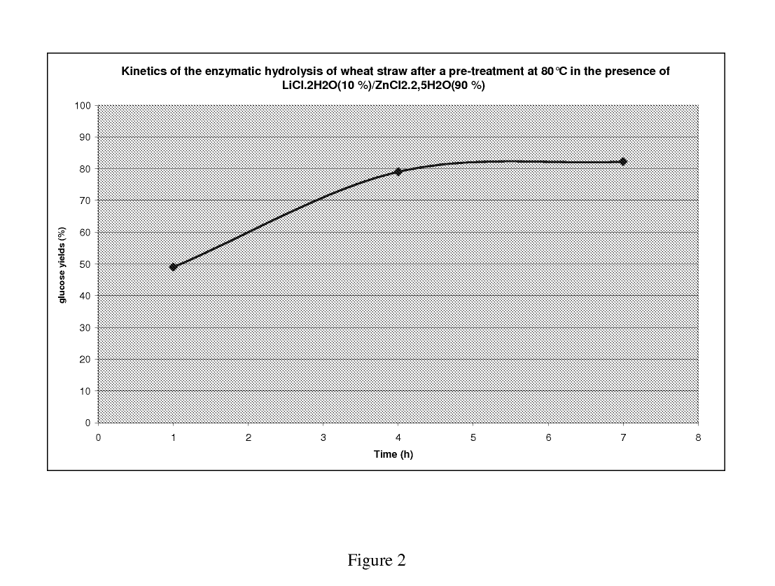

The protocol was identical to that of Example 1 apart from the fact that 38 g of LiCl.2H₂O (10%)/ZnCl₂.2.5 H₂O(90%) was used instead of the 38 g of LiCl.H₂O (98%)/ZnCl₂.2.5H₂O (2%) and that the pre-treatment was carried out at 80° C. The results of the enzymatic hydrolysis are shown in FIG. 2.

### Example 3

### Pre-Treatment of Wheat Straw with NaCl.6H₂O(10%)/ZnCl₂.2.5H₂O(90%) at 80° C. and Enzymatic Hydrolysis

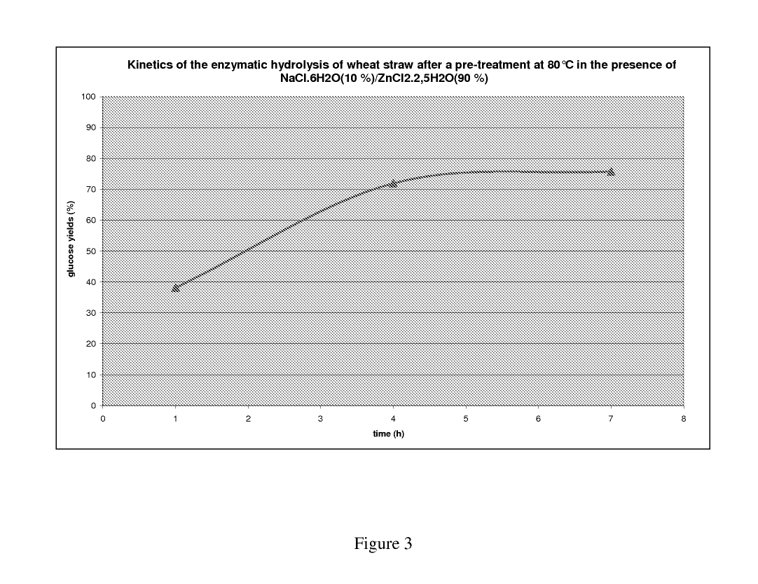

The protocol was identical to that of Example 1 apart from the fact that 38 g of NaCl.6H₂O(10%)/ZnCl₂.2.5H₂O(90%) was used instead of the 38 g of LiCl.H₂O(98%)/ZnCl₂.2.5H₂O(2%) and that the pre-treatment was carried out at 80° C. The results of the enzymatic hydrolysis are shown in FIG. 3.

### Example 4

### Pre-Treatment of Wheat Straw with NaCl.6H₂O(10%)/ZnCl₂.2.5H₂O(90%) Recycled at 80° C. and Enzymatic Hydrolysis

The centrifuging supernatants obtained during a pre-treatment as described in Example 3 were combined and concentrated in the rotary evaporator (Tmax=120° C.−P min=90 mbar) to produce 34.8 g of a residue containing 32.2 g of ZnCl₂.2.3H₂O and 1.9 g of NaCl.1.8 H₂O. The stoichiometry of the water with respect to the salts was adjusted by adding 2 g of water.

The mixture of recycled salts obtained thereby and 2 g of wheat straw (500-1000 μm) were placed in a 170 mL flask and stirred mechanically at 60° C. for 1 h in a Tornado© stirrer provided with a 6-position carousel (Radleys).

After this pre-treatment (step a) of the process of the invention), heating was halted and 80 mL of distilled water was rapidly added to the mixture: the pre-treated straw precipitated out. The suspension containing the mixture of salts, water and biomass was placed in a centrifuge tube and stirred at 9500 rpm for 10 minutes. The supernatant containing the inorganic salt was then separated from the solid. The operation was carried out twice by adding 80 mL of distilled water to the solid portion still present in the centrifuging tube.

11.2 g of solid with a dry matter content of 13% was recovered.

The solid recovered after precipitation and washing underwent enzymatic hydrolysis. Half of the recovered solution was placed in a 100 mL Schott flask. 5 mL of acetate buffer, and 10 mL of a 1% by weight solution of NaN₃ in water were added then made up to 100 g with distilled water. This solution was then left overnight at 50° C. for “activation”, at an agitation rate of 550 rpm in a STEM heater/shaker reaction station. Next, known quantities of enzyme were added to the solution:
XL508 Cellulases, 10 FPU per gram of dry matter;
NOVOZYM 188 β-glucosidases, 25 CBU per gram of dry matter.

The solution was then stirred at 400 rpm at 50° C., still in the heater/shaker reaction station, and samples were taken after 1 h, 4 h and 7 h. These samples were placed in centrifuge tubes and rapidly placed in an oil for 10 minutes at a temperature of 103° C. to neutralize the enzymatic activity. The centrifuge tubes were stored in a refrigerator at 4° C. to await glucose measurement. They were then diluted 5-fold with distilled water before being assayed using an Analox GL6 analytical apparatus known as a glucostat, which measures the concentration of glucose in aqueous solutions by enzymatic assay.

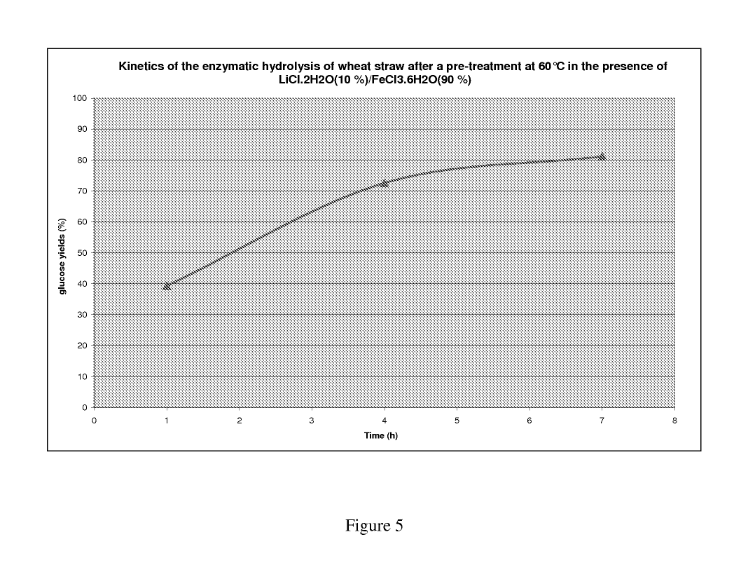

The results of the enzymatic hydrolysis are shown in FIG. 4. They are expressed as the glucose yield, defined as the ratio of the concentration of glucose in the solution to its maximum theoretical concentration as a function of the quantity of cellulose in the native wheat straw. This glucose yield thus represents the percentage of cellulose contained in the native substrate effectively transformed into glucose after the pre-treatment and enzymatic hydrolysis steps.

### Example 5

### Pre-Treatment of Wheat Straw with LiCl.2H₂O(10%)/FeCl₃.6H₂O(90%) at 60° C. and Enzymatic Hydrolysis

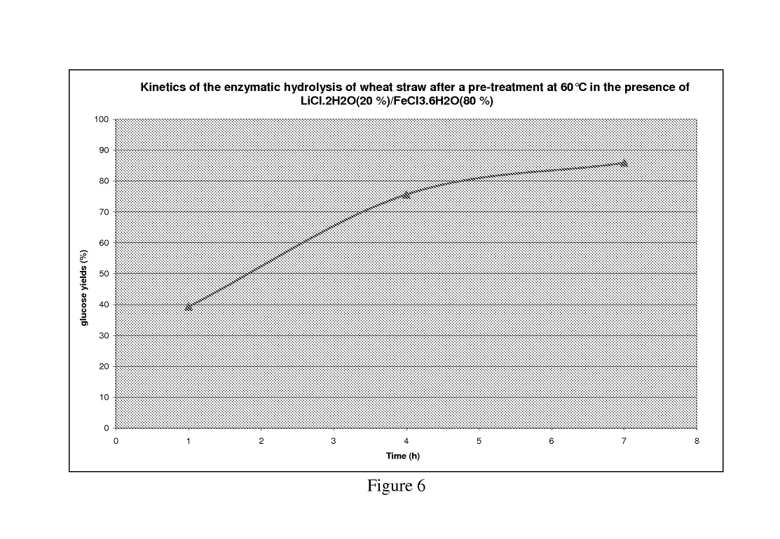

The protocol was identical to that of Example 1 apart from the fact that 38 g of LiCl.2H₂O(10%)/FeCl₃.6H₂O(90%) was used instead of the 38 g of LiCl.H₂O(98%)/ZnCl₂.2.5H₂O(2%) and that the pre-treatment was carried out at 60° C. The results of the enzymatic hydrolysis are shown in FIG. 5.

### Example 6

### Pre-Treatment of Wheat Straw with LiCl.2H₂O(20%)/FeCl₃.6H₂O(80%) at 60° C. and Enzymatic Hydrolysis

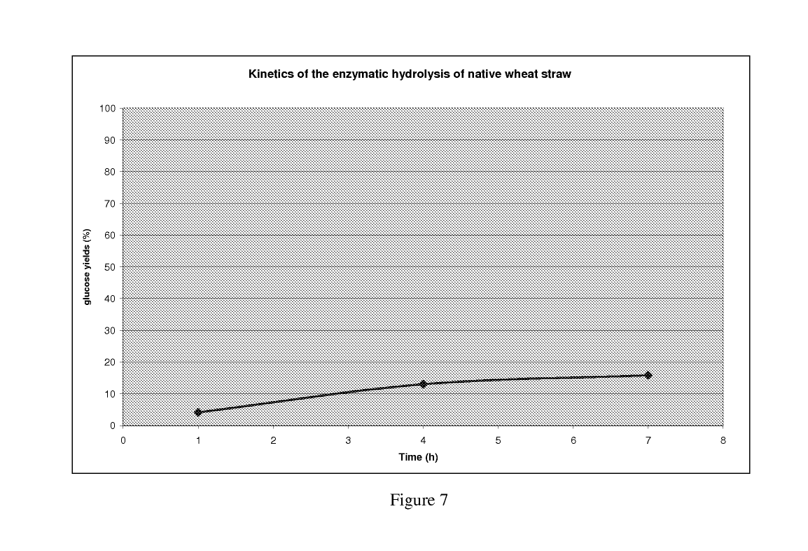

The protocol was identical to that of Example 1, apart from the fact that 38 g of LiCl.6H₂O(20%)/FeCl₃.6H₂O(80%) were used instead of 38 g of LiCl.H₂O(98%)/ZnCl₂.2.5 H₂O(2%) and that the pre-treatment was carried out at 60° C. The results of the enzymatic hydrolysis are shown in FIG. 6.

### Example 7

### Not in Accordance with the Invention

### Enzymatic Hydrolysis of Native Wheat Straw (No Pre-Treatment)

The protocol used for enzymatic hydrolysis was identical to that described in Example 1. The wheat straw was used in the native form, without any pre-treatment.

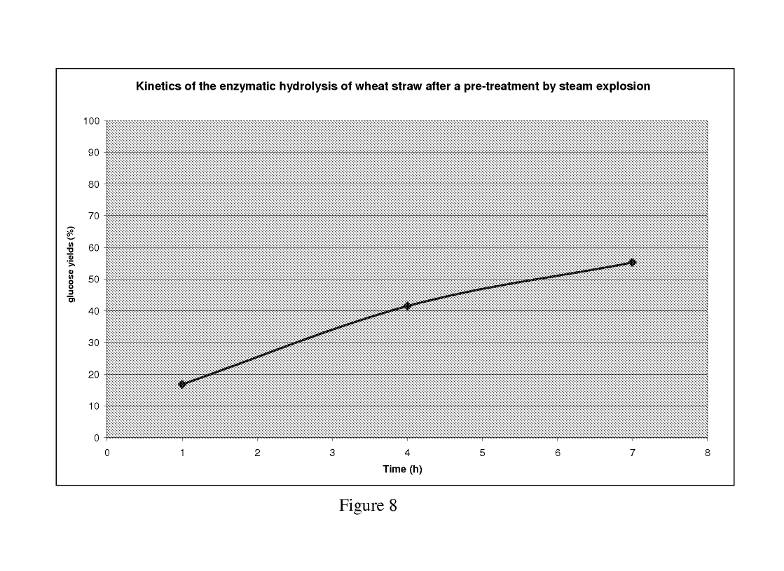

The results of the enzymatic hydrolysis are shown in FIG. 7.

### Example 8

### Not in Accordance with the Invention

### Enzymatic Hydrolysis of Wheat Straw Obtained from Steam Explosion Pre-Treatment

The protocol used for enzymatic hydrolysis was identical to that described in Example 1. The wheat straw used was pre-treated by steam explosion, in which pre-treatment the three steps below were carried out:
1/ impregnating the wheat straw with 0.1 N sulphuric acid for a minimum of 8 hours followed by draining and compressing (100 bar) the straw to obtain a solid with approximately 30% dry matter content;
2/ pre-treating the straw at 210° C. (18 bar) for 2.5 minutes then decompression;
3/ final treatment by compressing at 100 bar.

The results of the enzymatic hydrolysis are shown in FIG. 7.

From these various figures representing each of the examples, it can be seen that the process of the present invention can be used to rapidly obtain (after 1 hour of enzymatic hydrolysis) substantially higher yields of glucose than those obtained without pre-treatment, or even after pre-treatment of the steam explosion type, since in this case, the glucose yield after 1 hour was only approximately 20%.

In the same manner, the glucose yields obtained after 7 hours of hydrolysis were improved compared with those of the reference technology (steam explosion) when the cooking step was carried out using the process described in the present invention.

Excellent yields (more than 80% of the glucose yield) were obtained at moderate temperatures (of the order of 60° C.).

1. A process for the conversion of lignocellulosic biomass into monosaccharides, comprising at least:
a) a step for cooking the biomass in the presence or in the absence of an organic solvent in a medium comprising at least one hydrated organic salt with formula (1):
MXₙ.n′H₂O
in which X is an anion and M is a metal selected from groups 1 and 2 of the periodic classification of the elements, n is a whole number equal to 1 or 2 and n′ is in the range 0.5 to 6; mixed with at least one other metallic salt, which may or may not be hydrated, with general formula (2):
M′Yₘ.m′H₂O
in which Y is an anion, which may be identical to or different from X, and M′ is a metal selected from groups 3 to 13 of the periodic classification of the elements, m is a whole number in the range 1 to 6 and m′ is in the range 0 to 6;
b) a step for separating a solid fraction which has undergone step a);
c) a step for enzymatic hydrolysis of said solid fraction.

2. A process according to claim 1, in which the medium in which the cooking step is carried out is constituted by one or more hydrated inorganic salts with formula (1) mixed with at least one other metallic salt, which may or may not be hydrated, with formula (2).

3. A process according to claim 1, in which the anion X is a halide anion selected from Cl, F, Br and I, a perchlorate anion, a thiocyanate anion, a nitrate anion or an acetate anion.

4. A process according to claim 1, in which the anion Y, which may be identical to or different from the anion X, is selected from halides, nitrates, carboxylates, halogenocarboxylates, acetylacetonate, alcoholates, phenolates, optionally substituted, sulphates, alkylsulphates, phosphate, alkylphosphates, fluorosulphonate, alkylsulphonates, perfluoroalkylsulphonates, bis(perfluoroalkylsulphonyl)amides, arenesulphonates, optionally substituted with halogen or haloalkyl groups.

5. A process according to the claim 4, in which the anion Y is selected from fluoride, chloride, bromide, acetate and triflate anions.

6. A process according to claim 1, in which the metal M is selected from lithium, magnesium, calcium, potassium and sodium.

7. A process according to claim 1, in which the metal M′ is selected from iron, cobalt, nickel, copper, zinc, aluminium, indium and lanthanum.

8. A process according to claim 1, in which the cooking step is carried out at a temperature in the range −20° C. to 250° C., preferably in the range 20° C. to 160° C.

9. A process according to claim 1, in which the molar fraction of the hydrated inorganic salt with general formula (1) in the mixture of salts with formula (1) and (2) is in the range 0.05 to 1.

10. A process according to claim 1, in which the cooking period is in the range 0.5 minutes to 168 hours, preferably in the range 5 minutes to 4 hours.

11. A process according to claim 1, in which the lignocellulosic biomass is present in a quantity in the range 0.5% to 40% by weight of the total mass of the lignocellulosic biomass/hydrated inorganic salt/metallic salt mixture, preferably in a quantity in the range 3% to 25% by weight.

12. A process according to claim 1, in which the step for cooking the biomass may be carried out in a medium constituted by a mixture of different hydrated inorganic salts with formula (1) and/or different metallic salts with formula (2).

13. A process according to claim 1, in which the step for cooking the lignocellulosic biomass is carried out in the presence of an organic solvent selected from alcohols such as methanol, ethanol, n-propanol, isopropanol, n-butanol, isobutanol or tert-butanol, diols and polyols such as ethanediol, propanediol or glycerol, amino alcohols such as ethanolamine, diethanolamine or triethanolamine, ketones such as acetone or methyl ethyl ketone, carboxylic acids such as formic acid or acetic acid, esters such as ethyl acetate or isopropyl acetate, dimethylformamide, dimethylacetamide, dimethylsulphoxide, acetonitrile, and aromatic solvents such as benzene, toluene, xylenes, and alkanes.

14. A process according to claim 1, in which the step for separating the solid fraction is carried out by precipitation by adding at least one anti-solvent selected from water, alcohols such as methanol, ethanol, n-propanol, isopropanol, n-butanol, isobutanol or tert-butanol, diols and polyols such as ethanediol, propanediol or glycerol, amino alcohols such as ethanolamine, diethanolamine or triethanolamine, ketones such as acetone or methyl ethyl ketone, carboxylic acids such as formic acid or acetic acid, esters such as ethyl acetate or isopropyl acetate, dimethylformamide, dimethylacetamide, dimethylsulphoxide, and acetonitrile.

15. A process according to claim 1, in which the enzymatic hydrolysis is carried out at a temperature of the order of 40° C. to 60° C., at a pH in the range 4.5 to 5.5, for a period of 1 to 150 h.
